(19) United States
(12) Patent Application Publication
(10) Pub. No.: US 2025/0339353 A1
(43) Pub. Date: Nov. 6, 2025

(54) A HIGH SPF GEL COMPOSITION

(71) Applicant: Conopco, Inc., d/b/a UNILEVER, Englewood Cliffs, NJ (US)

(72) Inventors: Aditi Jayavant KULKARNI, Mumbai (IN); Praful Gulab Rao LAHORKAR, Bangalore (IN); Ankita Rutu PAWAR, Mumbai (IN); Rajkumar PERUMAL, Karnataka (IN); Ashish Anant VAIDYA, Bangalore (IN); Kalavathi RAMANAN, Chennai (IN)

(73) Assignee: Conopco, Inc., d/b/a UNILEVER, Englewood Cliffs, NJ (US)

(21) Appl. No.: 18/864,978

(22) PCT Filed: May 10, 2023
(86) PCT No.: PCT/EP2023/062520

§ 371 Date: Nov. 12, 2024

(30) Foreign Application Priority Data
May 13, 2022 (EP) 22173243.1

(51) Int. Cl. A61K 8/25; A61Q 17/04

(52) U.S. Cl. CPC A61K 8/25; A61Q 17/04; A61K 2800/412; A61K 2800/48; A61K 2800/524; A61K 2800/592; A61K 2800/651

(57) ABSTRACT

The present invention relates to a photoprotective personal care composition, which provides high sun-protection factor along with very good sensorial properties that the consumer expects when such a composition is applied on the skin. This is achieved through a composition comprising a combination of an oil-soluble sunscreen, a water-soluble sunscreen, a water phase thickener, an oil phase thickener, a humectant; and a select sensory modifier.

### FIELD OF THE INVENTION

The present invention relates to a photoprotective personal care composition, which provides high sun-protection factor (SPF). It more particularly relates to a high SPF gel composition which delivers very good sensorial properties that the consumer expects when such a composition is applied on the skin like low oiliness and good spreadability.

### BACKGROUND OF THE INVENTION

Solar radiation includes about 5% ultraviolet (UV) radiation, wavelength of which is between 200 nm and 400 nm. It is further divided into three regions: from 320 to 400 nm (UV-A), 280 to 320 nm (UV-B) and from 200 to 280 nm (UV-C). Substantial portion of UV-C radiation is absorbed by ozone. Scientific studies have indicated that exposure to UV-A and UV-B radiation for short period causes pigmentation, reddening of the skin and localized irritation, whereas continued or prolonged exposure can lead to sunburn, melanoma, formation of wrinkles and age spots. UV radiation is also thought to cause significant damage to hair. Therefore, it is desirable to protect the skin and other keratinous substrates of the human body from the harmful effects of both UV-A and UV-B radiation. This is delivered though cosmetic compositions which provide high SPF (Sun Protection Factor).

Various cosmetic preparations are reported for preventing and/or protecting the skin from harmful effects of ultraviolet radiation. Numerous organic sunscreen agents capable of absorbing UV-A rays are reported in the field of cosmetics amongst which a particularly useful sunscreen is t-butylmethoxydibenzoylmethane (also known as Avobenzone and sold as Parsol® 1789). A variety of UV-B sunscreens are used in personal care compositions for protection from UV-B radiation. Many cosmetic manufacturers prefer to include UV-A as well as UV-B sunscreens in photoprotective compositions to provide protection over the entire range of UV radiation.

One such patent from the present applicants is published as EP2575748B1 which discloses a composition which delivers high SPF. This publication only discloses use of certain oil soluble sunscreens. With the advent of many water-soluble sunscreens, the present inventors experimented with a combination of oil-soluble and water-soluble sunscreens to deliver better SPF. They also wanted to deliver it through a gel type composition which is generally liked by many consumers as it gives a light, less oily and pleasant skin feel when such compositions are applied on the skin. The present inventors during delivering the benefits of high SPF through a composition comprising both oil soluble as well as water soluble sunscreens found that when water soluble sunscreens (generally added in salt form) are included, it is difficult to stabilize them in such gel compositions i. e. it is difficult to provide a stable gel composition that comprises water soluble sunscreens. Thus, the present inventors not only had to stabilize the water-soluble sunscreens while ensuring the desired high SPF but also had to provide the desired tactile sensorial like low oiliness and good spreadability. With further extensive experimentation, the present inventors could ensure the delivery of the desired high SPF as well as the desired stability of the sunscreens by including therein select water phase thickener, certain type of oil phase thickener, and specific humectants.

However, the composition so prepared was seen to have poor sensory. By good sensory is meant that the composition gives a good moisturizing feel to the skin without being too oily. In other words, the consumers expect a moist feel on the skin while enjoying a matte (non-oily) appearance. Further, they expect other attributes like ease of spreading and a mild, gentle, and non-sticky feel on the skin. The inventors could ensure that the above desired sensorial could be delivered by including therein specific sensory modifiers. The high SPF so delivered, ensured that the skin is protected from darkening, and that blemishes and dark spots are reduced.

It is thus an object of the present invention to provide for a photoprotective personal care composition in gel form comprising both oil soluble and water-soluble sunscreens that not only gives high SPF but also delivers light, non-oily and non-sticky feel when applied on the skin. It is another object of the invention to provide such photoprotective personal care composition that is a stable gel composition.

### SUMMARY OF THE INVENTION

The first aspect of the present invention relates to a high SPF gel composition comprising:
(i) 0.1 to 25 wt % oil-soluble sunscreen;
(ii) 0.1 to 5 wt % water-soluble sunscreen;
(iii) 0.25 to 5 wt % of a water phase thickener selected from one or both of a polysaccharide gum and a synthetic polymer selected from ammoniumacryloyldimethyltaurate/VP copolymer, and ammonium acryloyldimethyltaurate/beheneth-25 methacrylate crosspolymer;
(iv) 0.5 to 10% of an oil phase thickener selected from one or more of an ester of C18 to C36 fatty acid, ester of C8 to C16 fatty acid; polymeric oil phase thickener and particle selected from organically modified clay, hydrophobically modified fumed silica and aerogels,
(v) 0.5 to 15 wt % humectant selected from glycerol or a glycol; and
(vi) 0.5 to 10 wt % of a sensory modifier selected from one or more of a modified starch, silica microsphere and a polymer of dimethicone/vinyldimethicone crosspolymer and silica.

Another aspect of the present invention relates to a method of providing photoprotection with a Sun Protection Factor (SPF) of at least 30 comprising a step of applying a composition of the first aspect on to the desired skin surface.

### DETAILED DESCRIPTION OF THE INVENTION

These and other aspects, features and advantages will become apparent to those of ordinary skill in the art from a reading of the following detailed description and the appended claims. For the avoidance of doubt, any feature of one aspect of the present invention may be utilized in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated. Except in the operating and comparative examples, or where otherwise explicitly indicated, all numbers in this description and claims indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated.

The photoprotective composition of the invention is meant to be used for personal care or for cosmetic use and could also be referred to as a personal care composition or a cosmetic composition. By a “personal care composition” as used herein, is meant to include a composition for topical application to sun-exposed areas of the skin and/or hair of humans. Such a composition may be classified as leave-on or rinse off, and includes any product applied to a human body for improving appearance, cleansing, odour control or general aesthetics. The composition of the present invention can be in the form of a liquid, or gel. Non-limiting examples of such sunscreen compositions include leave-on skin products like antiperspirants, deodorants, sunscreen gels and wash-off shampoos, conditioners, face wash and shower gels. The composition of the present invention is preferably a leave-on composition.

“Skin” as used herein is meant to include skin on the face and body (e.g., neck, chest, back, arms, underarms, hands, legs and scalp) and especially to the sun exposed parts thereof. Compositions of the invention is also of relevance to applications on any other keratinous substrates of the human body other than skin e.g., hair where products may be formulated with specific aim of improving photoprotection.

The compositions of the invention comprise 0.1 to 25% by weight oil-soluble sunscreen. By oil-soluble is meant those organic sunscreens which have a solubility in water of less than 10 g/l, preferably less than 5 g/l, more preferably less than 2 g/l. Oil-soluble sunscreens could be of the UV-B type or of the UV-A type. It is preferred that the composition in accordance with the invention comprises 0.1 to 20%, preferably 1 to 15% by weight of a UVB oil soluble sunscreen. It is further preferred that the composition of the invention comprises 0.1 to 5%, preferably 1 to 4% by weight of a UVA oil soluble sunscreen.

When an oil soluble UVB sunscreen is included, it is preferably one or more of octyl salicylate (Eusolex® OS), 3,3,5-trimethylclohexyl 2-hydroxybenzoate, ethylhexyl salicylate, 2-ethylhexyl 2-cyano-3,3-diphenyl-2-propenoate which is also known as octocrylene, 2-ethylhexyl-4-methoxycinnamate or octylmethoxycinnamate, ethylhexyl triazone, 3-benzylidene camphor, cinoxate, diethylhexyl butamido triazone, para amino benzoic acid (PABA), ethyl dihydroxypropyl PABA, ethylhexyl dimethoxy benzylidene dioxoimidazoline propionate, isoamyl p-methoxycinnamate, isopentyl trimethoxycinnamate trisiloxane, isopropyl benzyl salicylate, isopropyl methoxycinnamate, 4-methylbenzylidene camphor, PEG-25 PABA, and pentyl dimethyl PABA. More preferred oil-soluble UVB sunscreen is selected from one or more of octyl salicylate, 2-ethylhexyl 2-cyano-3,3-diphenyl-2-propenoate, and octylmethoxycinnamate.

Some of the well-known brands under which the above sunscreens are sold are Octisalate®, Homosalate®, Neo Heliopan®, Neo Heliopan® AV, Neo Heliopan® OS, Octocrylene® and Parsol® MCX. The oil-soluble UV-B sunscreen has Amax from 280 to 320 nm.

Composition of the invention may comprise an organic oil-soluble UVA sunscreen. When included, it is selected from one or more of a dibenzoylmethane compound, diethylamino hydroxybenzoyl hexyl benzoate and methyl anthranilate. A dibenzoyl methane compound is most preferred for an oil-soluble UVA-sunscreen. Suitable dibenzoyl methane compounds are selected from one of more of t-butylmethoxy dibenzoylmethane, 2-methyldibenzoylmethane, 4-methyl-dibenzoyl-ethane, 4-isopropyldibenzoyl-methane, 4-tert-butyldibenzoylmethane, 2,4-dimethyldibenzoylmethane, 2,5-dimethyldibenzoylmethane, 4,4′-diisopropyl-dibenzoylmethane, 2-methyl-5-isopropyl-4′-methoxydibenzoylmethane, 2-methyl-5-tert-butyl-4′-methoxy-dibenzoyl methane, 2,4-dimethyl-4′-methoxy dibenzoylmethane and 2,6-dimethyl-4-tert-butyl-4′-methoxydibenzoylmethane. The most preferred dibenzoyl methane compound is t-butylmethoxy dibenzoylmethane. Commercially available as Parsol 1789.

Composition of the invention may comprise both an organic oil soluble UVB and an organic UVA sunscreen. Alternately, there are certain oil soluble sunscreens which have both a UVA and UVB screening efficacy. It is selected from one or more bis-ethylhexyloxyphenol methoxyphenyl triazine, drometrizole trisiloxane, benzophenone and its derivatives. The most preferred one is bis-ethylhexyloxyphenol methoxyphenyl triazine, commercially available as Tinosorb S.

The composition of the invention comprises 0.1 to 5 wt % of a water-soluble sunscreen. By water soluble sunscreen, whether of the UVA type, or of the UVB type, is meant that the solubility in water of the sunscreen is higher than 10 g/l preferably higher than 50 g/l at 25° C.

The preferred water-soluble UVB sunscreen for use in the present invention is phenyl benzimidazole 5-sulphonic acid (PBSA), benzophenone-4 or benzylidene camphor sulfonic acid, preferably PBSA. PBSA also known as Ensulizole is commercially available as Eusolex 232 (from Merck KGaA). PBSA is also available under the brand names Neo Heliopan Hydro (from Symrise), Parsol HS (from DSM) and Sunsafe ES (from Uniproma). There are certain water-soluble sunscreens which have both a UVA screening efficacy as well as a UVB screening efficacy. Such sunscreens provide both UVA and UVB protection e.g. benzophenone-4 (sold as Sulisobenzone). However, for the purposes of the present invention benzophenone-4 may be included as a water-soluble UV-B sunscreen.

The preferred UVA sunscreen for use in the present invention is one or more of disodium phenyl dibenzimidazole tetrasulfonate (Neoheliopan AP) or terephthalylidene dicamphor sulfonic acid (TDSA). Of these, di sodium phenyl dibenzimidazole tetra sulfonate is preferred which is also known as bisdisulizole disodium. This is commercially available as Neo heliopan AP (from Symrise Shanghai Ltd) or as Sunsafe DPDT (from Uniproma)

Terephthalylidene dicamphor sulfonic acid (TDSA) is also known as Ecamsule. This is commercially available as Mexoryl SX (U.S. Pat. No. 4,585,597) by L'Oreal or Sunsafe TDSA (from Uniproma). It can be used as parent acid or its salts to deliver the desired benefit.

The composition preferably comprises 0.1 to 5 wt %, more preferably 1 to 4 wt % water soluble sunscreen. It is preferred that the composition comprises both a UVA and a UVB water-soluble sunscreen. Most preferred is to include one or both of 2-phenylbenzimidazole-5-sulfonic acid and di sodium phenyl dibenzimidazole tetra sulfonate.

The water-soluble sunscreens for inclusion in the composition of the present invention are generally commercially available in the acid form. When included in the composition in the acid form (pre neutralized forms) i.e having sulphonic acid group (—SO₃H) the composition additionally comprises a neutralising agent to convert the acid form into the salt form, in which form it is known to be active as a sunscreen with an exception to Mexoryl SX which can act as sunscreen with and without neutralization. When included, the neutralising agent is preferably in 0.05% to 4% more preferably 0.2% to 2% by weight of the composition. The neutralising agent is preferably an inorganic or an organic alkali. Organic alkali is preferably an amine such as triethanol amine or diethanol amine. The present inventors have observed that the inorganic alkali is especially preferred. Preferred are alkali metal hydroxides. Most preferred metal hydroxide for inclusion as neutralising agent in the composition of the invention are sodium hydroxide or potassium hydroxide. It is an especially challenging problem to stabilize the salt form of the water soluble sunscreen in a gel composition and yet get the desired enhancement of SPF and the desired tactile sensory properties. The present inventors have solved this by specifically selecting the claimed features (iii) to (vi) of the present invention viz. water phase thickener, oil phase thickener, the humectant and the sensory modifier.

The composition of the invention comprises a water phase thickener. The water phase thickener may be selected from one or more of (i) a polysaccharide gum and (ii) a synthetic polymer selected from (a) ammoniumacryloyldimethyltaurate/VP copolymer, and (b) ammonium acryloyldimethyltaurate/beheneth-25 methacrylate crosspolymer. Of the above a polysaccharide gum is most preferred. Preferred polysaccharide gums for inclusion in the composition of the invention are one or more of xanthan gum, guar gum, cellulose gum, tara gum, gellan gum, gum arabic, sclerotium gum, carrageenan gum, and locust bean gum. The most preferred polysaccharide gum is xanthan gum. Xanthan gum is commercially available as Keltrol CG-V from CP Kelco supplier. The synthetic polymers ammoniumacryloyldimethyltaurate/VP copolymer is commercially available as Aristoflex AVC and ammonium acryloyldimethyltaurate/beheneth-25 methacrylate crosspolymer as Aristoflex HMB. In an especially preferred aspect of the present invention both a polysaccharide gum e.g. xanthan gum and synthetic polymers Aristoflex AVC and Aristoflex HMB may be included. The water phase thickener is included in 0.25 to 5%, preferably 0.5 to 4%, most preferably 1 to 3% by weight of the composition.

The composition of the invention includes an oil phase thickener selected from one or more of an ester of C18 to C36 fatty acid, ester of C8 to C16 fatty acid; polymeric oil phase thickener and particle selected from organically modified clays, hydrophobically modified fumed silica and aerogels. Of these, an ester of C18 to C36 fatty acid is most preferred.

Suitable commercially available ester of C18 to C36 fatty acid are C18-36 Acid Triglyceride (Syncrowax HGLC), C18-36 Acid Glycol Ester (Syncrowax ERLC), Glyceryl tribehenate (Syncrowax HRC), Glyceryl hydroxystearate (Naturchem GMHS), Glyceryl tri (12-hydroxystearate) (Thixcin R), C16-18 triglyceride, C16-24 triglyceride (Cremeol HF-62-SPC). An especially preferred oil phase thickener for use in the composition of the invention is available as Syncrowax HGLC from Croda Personal Care. It has a melting point in the range of 60-70° C. which also acts as a structure builder, crystallinity modifier and Emulsion stabilizer.

Example of esters of C8-C16 fatty acid is Hydrogenated cocoglycerides (Softisan 100).

Polymeric oil thickeners which may be included are Sorbitol/Sebacic Acid Copolymer Behenate (Syncrowax ORM), Tribehenin (and) Sorbitol/Sebacic Acid Copolymer Behenate (Syncrowax OSW), Dextrin Palmitate (Dextrin Palmitate LDXP, HDXP and HDXP-KL), amide terminated polyamides (OleoCraft LP-20, MP-30, HP-31, and MP-32), ester terminated polyamides (Uniclear 100VG and C75V), Stearoyl Inulin (Rheopearl ISK2), Poly C10-30 alkyl acrylate (Intellimer IPA 13-6 Polymer), poly C10-30 alkyl acrylate-MP 48C (Intellimer IPA 13-1 Polymer), and PVP (FlexiThix™ thickener).

Particles which act as oil phase thickeners could be selected from one or more of an organically modified clay such as cationic surfactant with a Sepiolite, Montmorillonite, Hectorite or Bentonite clay, fumed Silica, hydrophobically modified fumed Silica, and Aerogels such as Dow Corning VM-2270 Aerogel Fine particles, Aerosil® R 972 (Silica Silyate-Evonik).

The oil phase thickener is preferably included in 0.5 to 10%, more preferably from 0.8% to 5% and even more preferably from 1% to about 3%, by weight of the composition.

The composition of the invention comprises a humectant selected from a glycol or glycerol. The glycol is preferably selected from one or more of one of propylene glycol, butylene glycol, and caprylyl glycol. The humectant for use in the present invention is one or both of glycerol and caprylyl glycol. The composition comprises humectant at 0.5 to 15%, preferably 1 to 10%, more preferably 1 to 8% by weight of the composition.

The composition comprises a sensory modifier selected from one or more of
a modified starch,
silica microsphere and
a polymer of dimethicone/vinyldimethicone crosspolymer and silica.

Of these, one or both of silica microspheres and the polymer are most preferred to be included in the composition.

The polymer for use as a sensory modifier is preferably dimethicone/vinyldimethicone crosspolymer (and) silica. Dimethicone/Vinyl Dimethicone Crosspolymer (and) Silica is available as DOWSIL™9701 Cosmetic Powder from Dow Corning and is also known as DC 9701. Silica microsphere is available as MSS-500/3H from Kobo.

Preferred modified starch is aluminium or sodium starch octenylsuccinate, more preferably aluminium starch octenylsuccinate. Aluminum Starch Octenylsuccinate is a chemically modified starch, in particular starches that have undergone esterification. Such aluminum starch Octenylsuccinate is commercially available from suppliers like Akzo Nobel or Nouryon under the names Dry Flo® Pure and Dry Flo® PC. A variety of other suppliers sell it under their own brand name. In cosmetics and personal care products, Aluminum Starch Octenylsuccinate is used in the formulation of lotions, powders, makeup and underarm deodorants. It is used as a thickener and binder, anticaking agent and oil absorbent. The composition may comprise aluminium starch octenylsuccinate which is further compounded with other ingredients to deliver other benefits. e.g. the ingredient having INCI name Aluminum Starch Octenylsuccinate (and) Acrylates Copolymer (and) Magnesium Carbonate may be included. It is available from Nouryon as NATRASORB® HFB. The natural content is 81% and the suggested applications include moisture resistant products, oil control products, anhydrous products, antiperspirant sticks, sunscreen, and dry shampoo for a variety of benefits including oil-absorption, mitigation of syneresis and fragrance-retention. The literature of the ingredient from Nouryon discloses that it is crosslinked aluminium starch octenyl succinate surface treated with acrylates copolymer and magnesium carbonate.

The sensory modifier is included in 0.5 to 10%, preferably 1 to 8%, more preferably 1 to 5% by weight of the composition. Of these, the polymer, when included is preferably at 0.2 to 3%, the silica microsphere when included is from 0.2 to 3% and the modified starch when included is from 0.5 to 4% by weight of the composition.

The composition of the invention is preferably substantially free of an emulsifier. Emulsifiers are generally surfactants or surface-active agents. Preferably, by substantially free of an emulsifier is meant that the emulsifier is included in less than or equal to 2 wt %, more preferably less than 2 wt %, even more preferably less than 1.5 wt %, further more preferably less than 1 wt % and most preferably less than 0.5 wt %, ideally absent from the composition of the invention. Alternatively, the composition preferably comprises 0 to 2 wt %, more preferably from 0.1 to 1.5 wt %, even more preferably from 0.25 to 1 wt % and still more preferably from 0.5 to 0.75 wt % of an emulsifier. It will be understood that the terms e.g. ‘substantially free of an emulsifier’ and ‘emulsifier free’ may be used interchangeably and they denote the same meaning.

Accordingly, the composition is preferably an emulsifier-free gel composition comprising:
(i) 0.1 to 25 wt % oil-soluble sunscreen;
(ii) 0.1 to 5 wt % water-soluble sunscreen;
(iii) 0.25 to 5 wt % of a water phase thickener selected from one or both of a polysaccharide gum and a synthetic polymer selected from ammoniumacryloyldimethyltaurate/VP copolymer, and ammonium acryloyldimethyltaurate/beheneth-25 methacrylate crosspolymer;
(iv) 0.5 to 10% of an oil phase thickener selected from one or more of an ester of C18 to C36 fatty acid, ester of C8 to C16 fatty acid; polymeric oil phase thickener and particle selected from organically modified clay, hydrophobically modified fumed silica and aerogels,
(v) 0.5 to 15 wt % humectant selected from glycerol or a glycol; and
(vi) 0.5 to 10 wt % of a sensory modifier selected from one or more of a modified starch, silica microsphere and a polymer of dimethicone/vinyldimethicone crosspolymer and silica.

Water is generally included in gel compositions as the cosmetically acceptable base. Water may be included in 10 to 70%, preferably 20 to 70%, furthermore preferably 20 to 65% by weight of the composition.

Preferably, the composition further comprises niacinamide. Preferably, the composition comprises niacinamide in an amount from 0.1 to 10 wt %, more preferably from 1 to 8 wt %, even more preferably from 2 to 7 wt % and further more preferably from 3 to 5 wt %.

In addition to the ingredients disclosed earlier, the compositions, may and preferably do include other ingredients in order to perform one or more functions of this invention also include a cosmetically acceptable carrier. Emollient materials may serve as cosmetically acceptable carriers. These may be in the form of silicone oils, natural or synthetic esters, and hydrocarbons. Amounts of the emollients may range anywhere from 0.1 to 25%, preferably between 1 and 10% by weight of the composition. Silicone oils may be divided into the volatile and nonvolatile variety. The term “volatile” as used herein refers to those materials which have a measurable vapor pressure at ambient temperature. Volatile silicone oils are preferably chosen from cyclic (cyclomethicone) or linear polydimethylsiloxanes containing from 3 to 9, preferably from 5 to 6, silicon atoms.

Colorants, opacifiers and abrasives may also be included in compositions of the present invention. Each of these substances may range from 0.05 to 5%, preferably between 0.1 and 3% by weight of the composition.

The composition of the present invention provides in-vitro Sun Protection Factor (SPF) of preferably at least 30, more preferably at least 40 and even more preferably at least 50.

### Method of Using Compositions

The invention also provides for a method of providing photoprotection with a Sun Protection Factor (SPF) of at least 30 comprising a step of applying a composition of the invention on to the desired skin surface. The composition according to the invention is intended primarily as a product for topical application to human skin, especially as an agent for protecting from solar radiation, and preventing or reducing pigmentation, the appearance of wrinkled or aged skin, or age spots. Preferably, the method is cosmetic/non-therapeutic.

In use, a small quantity of the composition, for example from 1 to 5 ml, is applied to exposed area of the skin, from a suitable container or applicator and, if necessary, it is then spread over and/or rubbed into the skin using the hand or fingers or a suitable device.

The individual Sun Protection Factor (SPF) for each subject is defined as the ratio of the amount of energy (dose, in units of J/m² or in seconds of exposure time) required to produce minimal erythema on protected skin to the amount of energy needed to produce minimal erythema on untreated skin calculated as follows:

$$SPF = \frac{MED Protected Skin}{MED Unprotected Control Site}$$

The SPF for the product is calculated by taking the arithmetic mean of the individual SPF values for all the subjects.

While the above summarizes the present invention, it will become apparent to those skilled in the art that modifications, variations, and alterations may be made without deviating from the scope and spirit of the present invention as described and claimed herein. The invention will now be further illustrated in the following non-limiting examples.

### EXAMPLES

### Example a, 1-3: Compositions within and Outside the Invention and its Effect in Delivering SPF, UVAPF and Sensorial Properties

Briefly, the compositions shown in tables below were prepared as follows:

glycerin, disodium EDTA, phenylbenzimidazole sulfonic acid (Ensulizole), sodium hydroxide (q.s. to pH 7) were added to water taken in a main vessel. The mixture was stirred until water phase become clear and heated to 70° C. Oil phase containing an oil phase thickener where applicable, caprylyl glycol, ethylhexyl salicylate, octocrylene, octyl methoxycinnamate (Parsol® MCX) and butyl methoxydibenzoylmethane (Parsol® 1789) were prepared in a separate beaker and heated to 70° C. Aristoflex AVC and Aristoflex HMB were added to the main vessel under homogenization. The water phase was allowed to become a clear gel. Thereafter, the oil phase was added to the main vessel under homogenization at 3500 rpm. The mixture was homogenized for 10 minutes followed by addition of xanthan gum which was pre-dispersed in 5 mL of water. DC 245 and where applicable MSS 500/3H were mixed and added into the main vessel and were mixed for 10 minutes. Where applicable, Dowsil 9701 was then added to the main vessel and mixing was continued. Once the main vessel reached below 40° C., ethyl alcohol, perfume, phenoxyethanol were added in the main vessel; and were mixed for 10 minutes. When the formulation attained room temperature and homogenous structure, it was removed from the homogenizer and stored in containers.

Table 1 shows the various compositions in gel form that were prepared.

TABLE 1

| | Example | Example | Example | Example |
|---|---|---|---|---|
| Ingredient | A, wt % | 1, wt % | 2, wt % | 3, wt % |
| Glycerin | 7.00 | 7.00 | 7.00 | 7.00 |
| Xanthan gum | 0.40 | 0.50 | 0.40 | 0.40 |
| (Keltrol CG-V) | | | | |
| Ensulizole | 3.00 | 3.00 | 3.00 | 3.00 |
| (Parsol HS) | | | | |
| Sodium hydroxide | 5.30 | 5.30 | 5.30 | 5.30 |
| 10% solution | | | | |
| Artistoflex AVC | 1.00 | 1.00 | 1.00 | 1.00 |
| Aristoflex HMB | 0.30 | 0.30 | 0.30 | 0.30 |
| Syncrowax HGLC | 1.00 | 2.00 | 1.00 | 1.00 |
| Parsol 1789 | 3.00 | 3.00 | 3.00 | 3.00 |
| Eusolex OS | 5.00 | 5.00 | 5.00 | 5.00 |
| Octocrylene | 6.00 | 6.00 | 6.00 | 6.00 |
| (Neoheliopan 303/ | | | | |
| Parsol 340) | | | | |
| Parsol MCX | 1.50 | 1.50 | 1.50 | 1.50 |
| MSS-500/3H^{#} | — | 1.50 | 1.50 | 1.50 |
| DC 9701 | — | 1.00 | 2.00 | 1.00 |
| Caprylyl glycol | 0.50 | 0.50 | 0.50 | 0.50 |
| DC245 | 2.50 | 2.50 | 2.50 | 2.50 |
| Dry Flo pure | — | — | — | 0.50 |
| Natrasorb HFB | — | — | — | 1.00 |
| Phenoxyethanol | 0.50 | 0.50 | 0.50 | 0.50 |
| Ethyl alcohol | 5.00 | 5.00 | 5.00 | 5.00 |
| Tertiary butyl | 0.15 | 0.15 | 0.15 | 0.15 |
| alcohol | | | | |
| Water | to 100 | to 100 | to 100 | to 100 |

^{#}MSS-500/3H is a silica microsphere available from Kobo which acts as a sensory modifier in the present invention

The invitro SPF and UVAPF values for the above samples were measured using the following procedure:

Thin film transmittance measurements were done using Labsphere's Ultraviolet transmittance analyzer. In this study, 70×70 mm PMMA plate with ˜6 μm roughness from Schönberg GmbH & Co were used. The percent transmittance of the various compositions was measured using a procedure as outlined below. 2 mg/cm² of sample was applied on PMMA plate, distributed uniformly using a syringe, and spread uniformly. The drying time for the PMMA plates was 30 minutes. After the drying time, sample plates were exposed to UV light and transmittance scan was recorded. This scan gives the transmittance as a function of wavelength (290-400 nm) for a given sample. For a single plate six to nine different spots were scanned. The same was repeated for 2 or 3 plates. The data reported is thus an average over 12-18 readings. The reference transmittance scan was obtained using a blank plate, with no sample on the PMMA plates with glycerin spread on it. The transmittance values were used to arrive at the SPF values using the UV-2000s application provided with the instrument.

Percentage of transmittance (and therefore the SPF) was measured invitro SPF at zero time (line 0 min). Same plate was exposed to solar simulated light and % of transmittance measured at designated time intervals from 0 and 60 min. Average absorbance at 310 & 355 nm was recorded to measure % UVB & UVA photostability respectively. UV was generated using solar simulator (Atlas kW system) using irradiance of 550 W m⁻², at a distance of ˜ 20 cm.

The SPF and UVAPF values are summarised in Table-2 below:

TABLE 2

| | Example A, | Example 1, | Example 2, | Example 3, |
|---|---|---|---|---|
| Invitro SPF | 64.4 | 101.6 | 101.2 | 107.2 |
| STD dev | 22.8 | 26.7 | 35.5 | 17.8 |
| Invitro UVAPF | 21.2 | 32.8 | 38.7 | 40.1 |
| STD dev | 6.5 | 8.1 | 9.7 | 6.3 |

The data in the table—2 above indicates that a composition outside the invention (Example-A) where no sensory modifiers are included provides for much lower SPF and UVAPF values as compared to compositions within the invention (Examples 1 to 3) where different combination of sensory modifiers were included at various amounts.

### Difference in Sensory Benefits on Use of Various Compositions:

Compositions as per the invention (Examples 1 to 3) were studied for differences in the sensory benefits they provided using the following procedure:

The study was conducted on a total of 50 females subjects. They were engaged in a product use study that evaluated the tactile performance of the test products. Each product was evaluated for five days. The study has a sequential monadic design. Digital feedback was captured using structured questionnaire to understand the product preferences like even skin tone, brighter/fairer skin, glow, sweating, comfort and freshness, feel on face post application (Oily/sticky/greasy), prevention of sun tanning matt feel, spreadability, quick absorption, light feel, moisturization, sticky feel, peeling etc.

Each panelist evaluated the samples for five days each. The overall preference of the samples is summarized in Table-3 below:

TABLE 3

| | | | |
|---|---|---|---|
| Product | Example 1 | Example 2 | Example 3 |

| | | |
|---|---|---|
| 70 | 66 | 50 |

The data in the table-3 above indicates that composition of Example 1 is most preferred followed by Example 2 and then Example-3.

The data indicates that Composition as per Example 1 which has higher amount (2%) of oil phase thickener (Syncrowax HGLC) and the Sensory modifiers like silica microsphere (MSS-500/3H) at 1.5% and polymer (DC 9701) at 1% is preferred over Example 2 which has lower amount (1%) of oil phase thickener, same amount of silica microspheres and higher amount of polymer (2%). Example 3 is least preferred which additionally has starch based sensory modifiers (Natrasorb HFB and Dry Flo pure) over that of Example-1.

In the qualitative attributes, all the samples (Example 1 to 3) were well appreciated for spreadability, quick absorption, light feel & moisturization.

Example 1 and Example 2 were significantly better than Example-3 for even skin tone, brighter skin, glow, sweating, and Comfort & Freshness.

Example 1 was significantly better than Example-3 for feel on face post application (Oily/sticky/greasy), prevention of sun tanning, lasting time, protection from sun, ultra matt feel & oil free look whereas it was comparable with Example-2.

The data in summary indicates that all three examples as per the invention (Examples 1 to 3) were significantly better than control sample in sun protection. In the sensory domain Example 1 was most preferred followed by Example 2 and Example 3.

Further, example B as shown in table 4 below was prepared and SPF and UVAPF were measured as described above.

TABLE 4

| Ingredients (wt %) | Example B (wt %) |
|---|---|
| Glycerin | 7 |
| Xanthan Gum | 0.4 |
| (Keltrol CG-V) | |
| Ensulizole | 3 |
| (Parsol HS) | |
| Sodium Hydroxide | 5.3 |
| 10% solution | |
| Aristoflex AVC | 1 |
| Aristoflex HMB | 0.3 |
| Syncrowax HGLC | — |
| Parsol 1789 | 3 |
| Eusolex OS | 5 |
| Octocrylene | 6 |
| Parsol MCX | 1.5 |
| MSS-500/3H | 1.5 |
| DC 9701 | 2 |
| Caprylyl Glycol | 0.5 |
| DC 245 | 2.5 |
| Phenoxy Ethanol | 0.5 |
| Alcohol | 5 |
| Perfume | 0.35 |
| Water | To 100 |

The SPF and UVAPF values obtained were summarized in table 5 below:

TABLE 5

| | Example B |
|---|---|
| In-vitro SPF | 67.3 |
| STD dev | 9.4 |
| UVAPF | 23.8 |
| STD dev | 1.3 |
| Stability @ | Unstable |
| 45° C.; 3 weeks | |
| Sensory | Gel texture with |
| | oil leakage |

The data in table 5 above showed that when an oil phase thickener was absent, it provided much lower SPF and UVAPF than that provided by the compositions as per the present invention (examples 1 to 3) that contained an oil phase thickener. It also resulted in an unstable composition as the composition exhibited phase separation after being incubated at 45° C. for 3 weeks.

Further, compositions as per the present invention (examples 4 and 5) and a comparative composition (example C) as shown in table 6 below were prepared; and SPF, UVAPF and stability of these composition was measured.

TABLE 6

| (wt %) | Example 4 | Example 5 | Example C |
|---|---|---|---|
| Glycerin | 7 | 7 | 7 |
| Xanthan Gum | 0.4 | 0.4 | 0.4 |
| (Keltrol CG-V) | | | |
| Ensulizole | 3 | 3 | 3 |
| (Parsol HS) | | | |
| Sodium | 5.3 | 5.3 | 5.3 |
| Hydroxide 10% | | | |
| Aristoflex AVC | 1 | 1 | 1 |
| Aristoflex HMB | 0.3 | 0.3 | 0.3 |
| Thickener | 1 | 1 | 1 |
| | (Syncrowax ® | (Syncrowax ® | (Sunhancer ™ |
| | HGLC) | ORM) | Eco SPF |
| | | | Booster) |
| Parsol 1789 | 3 | 3 | 3 |
| Eusolex OS | 5 | 5 | 5 |
| Octocrylene | 6 | 6 | 6 |
| Parsol MCX | 1.5 | 1.5 | 1.5 |
| MSS-500/3H | 1.5 | 1.5 | 1.5 |
| DC 9701 | 2 | 2 | 2 |
| Caprylyl Glycol | 0.5 | 0.5 | 0.5 |
| DC 245 | 2.5 | 2.5 | 2.5 |
| Perfume | 0.35 | 0.35 | 0.35 |
| Phenoxy | 0.5 | 0.5 | 0.5 |
| Ethanol | | | |
| Ethyl Alcohol | 5 | 5 | 5 |
| Water | To 100 | To 100 | To 100 |

TABLE 7

| | Example 4 | Example 5 | Example C |
|---|---|---|---|
| SPF data | 109.2 | 91.5 | 104.1 |
| STD dev | 3.4 | 3.3 | 3.6 |
| UVAPF | 35 | 27.4 | 31.8 |
| STD dev | 5.9 | 0.7 | 1.6 |
| Stability @ | Stable | Stable | Unstable |
| 45° C.; 3 weeks | | | |

The data in table 7 above showed that when an oil phase thickener as per the present invention, i. e. an ester of C18 to C36 fatty acid (syncrowax HGLC) was used, a stable gel composition that provided high SPF and UVAPF was obtained (example 4). The data further showed that when an oil phase thickener of another type as per the present invention, i. e. polymeric oil phase thickener (sorbitol/sebacic acid copolymer behenate; available as Syncrowax® ORM (example 5)) was used, high SPF and UVAPF were obtained in a stable gel composition. Lastly, the data also showed that when an oil phase thickener that is not according to the invention, i. e. carnauba wax and rice bran wax (Sunhancer™ Eco SPF Booster as shown in example D) was used, it resulted in an unstable composition.

1. A high SPF gel composition comprising:
(i) from 0.1 to 25 wt % of an oil-soluble sunscreen,
(ii) from 0.1 to 5 wt % of a water-soluble sunscreen, wherein the water-soluble sunscreen is phenylbenzimidazole sulfonic acid;
(iii) from 0.25 to 5 wt % of a water phase thickener selected from a polysaccharide gum, a synthetic polymer, or combinations thereof, wherein the synthetic polymer is selected from ammoniumacryloyldimethyltaurate/VP copolymer, ammonium acryloyldimethyltaurate/beheneth-25 methacrylate crosspolymer, or combinations thereof;
(iv) from 0.5 to 10% of an oil phase thickener selected from an ester of C18 to C36 fatty acid, ester of C8 to C16 fatty acid, polymeric oil phase thickener, and particles, or combinations thereof, wherein the particles are selected from organically modified clays, hydrophobically modified fumed silica, aerogels, or combinations thereof;
(v) from 0.5 to 15 wt % of a humectant selected from glycerol, a glycol, or a combination thereof; and
(vi) from 0.5 to 10 wt % of a sensory modifier selected from a modified starch, silica microsphere, a polymer of dimethicone/vinyldimethicone crosspolymer and silica, or combinations thereof,
wherein a ratio of oil-soluble sunscreen to water-soluble sunscreen is about 5:1.

2. The composition as claimed in claim 1, wherein the oil-soluble sunscreen is selected from a UV-B sunscreen selected from octyl salicylate, 2-ethylhexyl 2-cyano-3,3-diphenyl-2-propenoate, octylmethoxycinnamate, or combinations thereof; or a UV-A sunscreen selected from a dibenzoylmethane compound.

3. (canceled)

4. The composition as claimed in claim 1, wherein the composition comprises from 0.1 to 20 wt % of the oil-soluble UVB sunscreen.

5. (canceled)

6. The composition as claimed in claim 1, wherein the polysaccharide gum is xanthan gum.

7. The composition as claimed in claim 1, wherein the glycol is selected from propylene glycol, butylene glycol, caprylyl glycol, or combinations thereof.

8. The composition as claimed in claim 1, wherein the sensory modifier is selected from the silica microsphere, the polymer of dimethicone/vinyldimethicone crosspolymer and silica, or combinations thereof.

9. The composition as claimed in claim 1, wherein the composition comprises from 10 to 70 wt % of water.

10. The composition as claimed in claim 1, wherein the composition is substantially free of an emulsifier.

11. The composition as claimed in claim 1, wherein the composition provides an in-vitro Sun Protection Factor of at least 30.

12. A method of providing photoprotection with a Sun Protection Factor of at least 30 comprising: applying the composition as claimed in claim 1 on to a desired skin surface.

13. The composition as claimed in claim 1, wherein composition comprises from 1 to 3.5 wt % of the sensory modifier, wherein the sensory modifier is the silica microsphere and the polymer of dimethicone/vinyldimethicone crosspolymer and silica.

14. The composition as claimed in claim 1, further comprising a volatile silicone oil.

15. The composition as claimed in claim 1, further comprising niacinamide.

16. The composition as claimed in claim 1, further comprising a preservative, wherein the preservative is phenoxyethanol.

17. A high SPF gel composition comprising:
(i) from 1 to 15 wt % of an oil-soluble UVB sunscreen, wherein the oil-soluble UVB sunscreen is t-butylmethoxy dibenzoylmethane, octyl salicylate, octocrylene,
(ii) from 1 to 4 wt % of an oil-soluble UVA sunscreen, wherein the oil-soluble UVA sunscreen is octylmethoxycinnamate;
(iii) from 1 to 4 wt % of a water-soluble sunscreen, wherein the water-soluble sunscreen is phenylbenzimidazole sulfonic acid;
(iv) from 1 to 3 wt % of a water phase thickener wherein the water phase thickener is xanthan gum, ammoniumacryloyldimethyltaurate/VP copolymer, and ammonium acryloyldimethyltaurate/beheneth-25 methacrylate crosspolymer;
(v) from 1 to 3% of an oil phase thickener wherein the oil phase thickener is a C18-36 acid triglyceride;
(vi) from 1 to 8 wt % humectant, wherein the humectant is glycerol and caprylyl glycol; and
(vii) from 1 to 5 wt % of a sensory modifier wherein the sensory modifier is a silica microsphere and a polymer of dimethicone/vinyldimethicone crosspolymer and silica, and optionally, a starch,
wherein a ratio of oil-soluble UVA and UVB sunscreen to water-soluble sunscreen is about 5:1.

18. The composition as claimed in claim 17, wherein a weight ratio of oil-soluble UVB sunscreen to the oil-soluble UVA sunscreen is 9.3 to 1.

19. The composition as claimed in claim 17, wherein the starch is aluminum starch octenylsuccinate.

20. The composition as claimed in claim 17, wherein the starch is aluminum starch octenylsuccinate and Acrylates Copolymer and Magnesium Carbonate.
